(19) United States
(12) Patent Application Publication
(10) Pub. No.: US 2023/0073614 A1
(43) Pub. Date: Mar. 9, 2023

(54) METHODS AND DEVICES FOR FILTERING CELL CULTURE MEDIA

(71) Applicants: Yissum Research Development Company of the Hebrew University of Jerusalem Ltd., Jerusalem (IL); Future Meat Technologies Ltd., Rehovot (IL)

(72) Inventors: Yaakov Nahmias, Mevaseret Zion (IL); Guy Wissotsky, Rishon LeZion (IL)

(73) Assignees: Yissum Research Development Company of the Hebrew University of Jerusalem Ltd., Jerusalem (IL); Future Meat Technologies Ltd., Rehovot (IL)

(21) Appl. No.: 17/759,707

(22) PCT Filed: Jan. 29, 2021
(86) PCT No.: PCT/IB2021/050728

§ 371 Date: Jul. 28, 2022

Related U.S. Application Data
(60) Provisional application No. 62/967,851, filed on Jan. 30, 2020.

(51) Int. Cl. B01D 15/36; B01D 61/22; B01D 63/02; C12M 1/00

(52) U.S. Cl. CPC B01D 15/361; B01D 61/22; B01D 63/02; C12M 29/18; C12M 47/02; B01D 2311/2626

(57) ABSTRACT

The present disclosure provides, in part, a receptacle for filtering a liquid. The receptacle comprises a plurality of hollow fibers extending the length of the receptacle and at least one solid absorbent material occupying a space between the plurality of hollow fibers. Each hollow fiber comprises at least one opening and a lumen defined by the walls thereof, allowing the liquid to flow through. The hollow fiber walls have a porosity profile selective to passage of waste materials contained in the liquid from the lumen to the solid absorbent material(s), thereby filtering the liquid. Also provided is a system as well as a method for filtering and recycling a cell culture medium.

### CROSS-REFERENCE TO RELATED APPLICATIONS

This application claims the benefit of priority to U.S. Provisional Patent Application No. 62/967,851, filed on Jan. 30, 2020, which is incorporated herein by reference in its entirety.

### FIELD OF THE INVENTION

The present invention generally relates to liquid filtering and recycling. More specifically, the present invention relates to methods and devices for filtering waste materials from fluids such as cell culture media and/or recycling the cell culture media.

### BACKGROUND

There are basically two main processes for large scale biological manufacturing of cells, proteins, or vaccines: either fed-batch or perfusion. In fed-batch process, cells grow in bioreactors with volumes as large as 25,000 liters and are continuously fed with nutrients until toxins reach a threshold (usually ammonia of 5 mM) and cells reach densities of up to 30 million cells/ml. In perfusion process, a medium is continuously replaced by filtering the cell suspension through a membrane (usually a hollow fiber membrane). This allows the toxins to be washed away, while allowing the cells to reach densities of up to 270 million cells/ml with bioreactors as big as 5,000 liters. However, the perfusion process wastes dozens of vessel volumes, making production as expensive as fed-batch process (albeit with 30% less expensive factory).

U.S. Pat. No. 5,071,561 discloses a method and apparatus for removing ammonia from cell cultures by contacting an aqueous culture medium with one side of a supported-fluid membrane wherein the support is a microporous hydrophobic polymeric membrane matrix; and maintaining a strip solution in contact with the other side of said membrane. Fidel Rey et al. (Cytotechnology 6: 121-130; 1991) discloses selective removal of ammonia from animal cell culture by utilizing a zeolite packed bed.

There is a need for an improved system or process for effectively filtering waste materials from cell culture media thus for large scale biological manufacturing of cells, proteins, or vaccines. The present invention fulfills this long-standing need.

### SUMMARY OF THE INVENTION

Disclosed herein are various filtering devices or systems for separating essential materials from waste materials in a liquid medium. While these devices or systems may be used for treating a vast array of liquid formulations or compositions, the present disclosure focuses on using these systems as efficient and simple ways to separate waste components from essentials of cell culture media.

One aspect of the present disclosure provides a receptacle for filtering a liquid. Such receptacle comprises a plurality of hollow fibers extending the length of the receptacle and at least one solid absorbent material occupying a space between the plurality of hollow fibers. Each hollow fiber comprises at least one opening and a lumen defined by the walls of the hollow fiber, which have a porosity profile selective to passage of waste materials contained in the liquid from the lumen to the at least one solid absorbent material. When the liquid flows along the lumen, it gets filtered.

In some embodiments, the at least one solid absorbent material is in a liquid environment having a pH ≥7 for effective interactions with the waste materials.

In some embodiments, the liquid is a cell culture medium comprising one or more materials selected from the group consisting of cells, tissues, nutrients, supplements, feeds, amino acids, peptides, proteins, vitamins, polyamines, sugars, carbohydrates, lipids, nucleic acids, hormones, fatty acids, trace materials and waste materials.

In some embodiments, the waste materials interfere with desired cell growth and/or desired cell differentiation, which include, but are not limited to, ammonia, lactate, toxins and sodium salts. In some embodiments, the waste materials have a molecular weight of no greater than 60 kDa.

In some embodiments, the cell culture medium contains tissues cultured for antibody production, growth factor production, or cultured meat production. While the waste materials are removed from the cell culture medium, any produced antibodies, produced growth factors, and produced cultured meat are kept in the cell culture medium.

In some embodiments, the porosity profile of the hollow fiber walls is configured to provide an average pore size and pore density that only permits passage of molecules that are smaller than 60 kDa. In some embodiments, the pore density is at least 10% of the wall surface of each hollow fiber.

In some embodiments, the at least one solid absorbent material is a microporous aluminosilicate material, an activated carbon, an ion-exchange resin, a charged polymer, a silica gel, a clay material, a resin material, or a combination thereof.

In some embodiments, the at least one solid absorbent material is a resin material selected from the group consisting of polyester resins, phenolic resins, alkyd resins, polycarbonate resins, polyamide resins, polyurethane resins, silicone resins, epoxy resins, polyethylene resins, polypropylene resins, acrylic resin resins and polystyrene resins.

In some embodiments, the waste materials comprise ammonia and the solid absorbent material or the waste treatment material comprises an aluminosilicate material. In some embodiments, the solid absorbent material is or comprises clinoptilolite.

In some embodiments, the waste molecules comprise lactate and the solid absorbent material or the waste treatment material comprises an ion-exchange resin. In some embodiments, the solid absorbent material is Amberlite®. In some embodiments, the solid absorbent material is or comprises Amberlite® IRA-400.

In some embodiments, the waste molecules comprise amphiphilic toxins and the solid absorbent material or the waste treatment material comprises carbon. In some embodiments, the solid absorbent material is or comprises activated carbon.

In some embodiments, the waste molecules comprise excess sodium ions and the solid absorbent material or the waste treatment material comprises ion-exchange resin. In some embodiments, the solid absorbent material is Amberlite®. In some embodiments, the solid absorbent material is or comprises Amberlite® 252RFH.

Another aspect of the present disclosure provides a system for filtering a cell culture medium. Such system comprises at least one receptacle described above and herein, means for flowing the cell culture medium through the plurality of hollow fibers of the receptacle, means for circulating the cell culture medium, and a bioreactor.

In some embodiments, the means for flowing the cell culture medium is a pump.

In some embodiments, the filtering system may further comprise at least one sensor configured to record values of at least one parameter related to the flow of the cell culture medium through the receptacle and/or the content of the cell culture medium. In some embodiments, the system may still further comprise a controller that is electrically connected to the pump and the at least one sensor. The controller is configured in such a way as to activate the pump based on signals received from the at least one sensor.

In some embodiments, the filtering system may further comprise at least one flow adaptor configured to fluidically connect the at least one receptacle to a recycling system.

In some embodiments, the recycling system is an alternating tangential flow (ATF) system, a Tangential Flow Filtration (TFF) system, a fed-batch culturing system, or a variation thereof.

In some embodiments, the filtering system comprises two or more receptacles that are fluidically connected to a recycling system. In some embodiments, the two or more receptacles are fluidically connected in a row or in parallel to each other. In some embodiments, the two or more receptacles are configured to treat different waste materials. In some embodiments, the two or more receptacles are configured to remove and/or deactivate two or more different waste materials.

In some embodiments, each of the two or more receptacles comprises a mixture of two or more solid absorbing materials for treatment of two or more different waste materials. In some embodiments, each of the two or more solid absorbing materials in the mixture is packed separately at different compartments inside each receptacle.

In some embodiments, the filtering system further comprises a passive flow receptacle. In some embodiments, the passive flow receptacle is configured to recycle the cell culture medium by osmosis or diffusion. In some embodiments, the passive flow receptacle is non-removably integrated with a cell culture container, or is removably placed inside a cell culture container or removably attached to an inner wall of a cell culture container. In some embodiments, the cell culture container is a cell culture plate, a cell culture flask, or a cell culture bioreactor.

In some embodiments, the cell culture medium is a suspension containing animal cells, and the suspension is perfused into the plurality of hollow fibers by a pump. In some embodiments, the pump is a positive displacement pump that pushes the suspension through the plurality of hollow fibers or alternates between pushing the suspension into the plurality of hollow fibers and drawing the suspension out into the bioreactor.

Still another aspect of the present disclosure provides a method for filtering a cell culture medium. This method comprises flowing the cell culture medium through a receptacle described above and herein, and then passing waste molecules through the walls of the plurality of hollow fibers to at least one solid absorbent material present outside the lumen and at a space between the plurality of hollow fibers. In this method, the nutrients are retained in the lumen, while the waste molecules leave the lumen and pass through the hollow fiber walls to reach at least one solid absorbent material that is in a liquid environment having a pH≥7.

In some embodiments, the method described above and herein may further comprise collecting the cell culture medium from the at least one opening, and re-flowing it through the plurality of hollow fibers one or more times, thereby recycling the cell culture medium.

In some embodiments, the method described above and herein involves a single pump.

In some embodiments, the method described above and herein does not involve active pumping. In some embodiments, the method involves passive penetration of the waste molecules through the walls of the plurality of hollow fibers.

In some embodiments, the method described above and herein involves the waste molecules ammonia and/or lactate.

In some embodiments, the method described above and herein is used to grow cultured meat.

Some aspects of the present disclosure provides a method for producing cultured tissues. This method comprises culturing tissues in a cell culture medium comprising nutrients and waste molecules; and filtering the cell culture medium according to the methods of filtering cell culture medium disclosed above and herein, to reduce the amount of waste molecules from the medium.

In some embodiments, the cultured tissues are used to produce cultured meat.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to better understand the subject matter that is disclosed herein and to exemplify how it may be carried out in practice, embodiments will now be described, by way of non-limiting example only, with reference to the accompanying drawings, in which:

FIG. 1 is a flow chart of a general process for removing waste from a culture medium while retaining nutrients.

FIG. 2 is a schematic representation of fluid recycling in a culture medium.

FIG. 3 is a block diagram of a culture medium recycling device.

FIGS. 4A and 4B are schematic representations of culture medium recycling using a culture medium recycling device.

FIG. 5 is a schematic representation of culture medium recycling using a culture medium recycling device comprising at least one hollow fiber membrane in tangential flow filtration (TFF) mode.

FIG. 6 is a schematic representation of a system for fluid, e.g., a culture medium, recycling.

FIG. 7 is a flow chart of closed loop culture medium recycling process.

FIG. 8A-8B depict different types of recycling devices. FIG. 8A is a schematic representation of at least one removably assembled recycling device added to a culture medium container. FIG. 8B is a schematic representation of at least one non-removable recycling device which is an integral part of a culture medium container.

FIGS. 9A and 9B are graphs showing accumulation of ammonia in a culture medium (FIG. 9A) and removal of ammonia from the culture media (FIG. 9B).

FIG. 10 is a bar graph showing cell viability measurement following exposure to toxic ammonia concentration with or without passing the cell suspension through hollow fiber packed with zeolite, demonstrating cell survival following ammonia absorption.

FIG. 11 is a bar graph showing absorbance of lactate to a resin.

FIG. 12 provides an indication of optimal lactate binding in both dextran and polylysine coatings without residual binding of glucose.

### DETAILED DESCRIPTION

For the purposes of promoting an understanding of the principles of the present disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

The terminology used herein is for the purpose of describing particular embodiments only and is not intended to be limiting of the invention.

As used herein, the term “medium” or “cell culture medium” encompasses any such medium as known in the art, including cell suspensions, blood and compositions comprising ingredients of biological origin. Such media and cultures may contain cells (mammalian cells, chicken cells, crustacean cells, fish cells and other cells), blood components, nutrients, supplements and feeds, amino acids, peptides, proteins and growth factors (such as albumin, catalase, transferrin, fibroblast growth factor (FGF), and others), vitamins, polyamines, sugars, carbohydrates, lipids, nucleic acids, hormones, fatty acids, trace materials, certain salts (such as potassium salts, calcium salts, magnesium salts), as well as waste materials such as ammonia, lactate, toxins and sodium salts. The medium is typically an aqueous based solution that promotes the desired cellular activity, such as viability, growth, proliferation, differentiation of the cells cultured in the medium. The pH of a culture medium should be suitable to the microorganisms that will be grown. Most bacteria grow in pH 6.5-7.0 while most animal cells thrive in pH 7.2-7.4.

As used herein, the terms “waste material(s)” and “waste molecule(s)” are interchangeable. These are any materials/molecules that interfere with desired growth and/or desired differentiation of the cells that are cultured in a cell culture medium, e.g., inhibit cell growth and/or differentiation or induce cell death. These materials/molecules are usually selected amongst minerals (mainly sodium salts) and small molecules (low molecular weight molecules). By way of non-limiting examples, the waste materials/molecules include, but are not limited to, ammonia, lactate, toxins and sodium salts.

As used herein, “hollow fibers” are elongated tubular membranes which may be specifically prepared from polymeric materials or other materials, or alternatively, obtained commercially. By way of non-limiting examples, hollow fibers and systems employing the same that can be used, modified or adapted for use in accordance with the present disclosure include those disclosed in U.S. Pat. Nos. 9,738,918; 9,593,359; 9,574,977; 9,534,989; 9,446,354; 9,295,824; 8,956,880; 8,758,623; 8,726,744; 8,677,839; 8,677,840; 8,584,536; 8,584,535; and 8,110,112, each of which is incorporated herein by reference.

As used herein, the terms “solid absorbent material(s)” and “waste treatment material(s)” are interchangeable. These are the material(s) present outside the lumen and at a space between the plurality of the hollow fibers of the receptacle. Suitable solid absorbent material or waste treatment material includes, but is not limited to, a microporous aluminosilicate material, an activated carbon, an ion-exchange resin, a charged polymer, a silica gel, a clay material, a resin material, and a combination thereof. Depending on the type of waste materials to be removed from the cell culture medium, different solid absorbent materials or waste treatment materials can be used.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

As disclosed herein, a cell culture medium containing cells in suspension is passed through a collection of elongated hollow fibers that are packed within a solid absorbent material. The elongated hollow fibers allow size-selective filtration through porous walls thereof, whereby nutrients and other essentials are prevented from passing through the hollow fiber walls and are thus maintained within the lumen formed by the hollow fiber walls, whereas waste materials are permitted to cross the porous walls and become absorbed in the solid absorbent material. Depending on the nature and amount of the waste materials, the interaction thereof with the solid absorbent material may be reversible or irreversible and may involve also chemical interaction which converts the waste materials to one or more other materials whose association with the solid absorbent material may be improved or irreversible.

In some embodiments, the cell culture medium is passed through the collection of hollow fibers once or multiple times, permitting size-selective filtration. In some embodiments, the cell culture medium is allowed to flow in the hollow fibers' lumen under pressure and other conditions which permit efficient filtration. Irrespective of the means by which the filtration is carried out, a superior and efficient separation is achieved due to (1) the hollow fibers having a porosity profile selective to passage of substantially only waste materials are used; (2) the porosity profile including pore diameters is effective in preventing passage of proteins, e.g., albumin, while permitting facile passage of waste materials to the solid absorbent material; (3) the solid absorbent material is selected to interact with or generally retain the waste materials, thereby substantially preventing the waste materials from flowing back into the hollow fibers' lumen; (4) the solid absorbent material is operable at pH above or equal to 7; and (5) an acidic cell culture medium is not used to capture or interact with basic waste materials, e.g., ammonia, thereby avoiding the basic waste materials from flowing back into the hollow fibers' lumen and thus diminishing the need to adjust the pH of the cell culture medium in the hollow fibers' lumen.

One aspect of the present disclosure provides a receptacle for filtering a liquid. Such receptacle comprises a plurality of hollow fibers extending the length of the receptacle and at least one solid absorbent material occupying a space between the plurality of hollow fibers. Each hollow fiber comprises at least one opening and a lumen formed by the walls of the hollow fibers. The hollow fibers' walls have a porosity profile selective to passage of waste materials contained in the liquid from the lumen to the at least one solid absorbent material, thereby filtering the liquid when it flows along the lumen.

In some embodiments, the at least one solid absorbent material is in a liquid environment of pH≥7. Such environment renders the receptacle effective in capturing waste materials that are passed through the hollow fiber's walls.

In some embodiments, the waste materials have a molecular weight of no greater than 60 kDa, e.g., no greater than 55 kDa, no greater than 50 kDa, no greater than 45 kDa, no greater than 40 kDa, no greater than 35 kDa, no greater than 30 kDa, no greater than 25 kDa, no greater than 20 kDa, no greater than 15 kDa, or no greater than 10 kDa.

In some embodiments, each hollow fiber comprises a first opening and a second opening. Accordingly, there is provided a filtering receptacle comprising a plurality of hollow fibers extending the length of the receptacle and at least one solid absorbent material occupying a space between the plurality of hollow fibers. Each hollow fiber has a first opening and a second opening and a lumen extending between the first and second openings. The lumen has a volume defined by the walls of the hollow fibers, permitting liquid communication between the first and second openings. The walls have a porosity profile selective to passage of waste materials contained in the liquid from the lumen volume to the at least one solid absorbent material. In some embodiments, the solid absorbent material is in a liquid environment of pH≥7.

In some embodiments, there is provided a filtering receptacle having a first face and a second face and comprising at least one solid absorbent material in an environment of pH≥7. The solid absorbent material(s) embedding a plurality of hollow fibers extend a length defined by the distance between the first and second faces of the receptacle. The hollow fibers have porous walls permitting irreversible passage of waste materials from a lumen of each of the hollow fibers to the solid absorbent material(s), wherein the receptacle is configured to permit flow of a liquid between the first face and the second face.

In some embodiments, there is provided a filtering receptacle having a first face and a second face and comprising at least one solid absorbent material having a pH≥7. The solid absorbent material(s) embedding a plurality of hollow fibers extend a length defined by the distance between the first and second faces of the receptacle. The hollow fibers having porous walls permitting passage of molecules having a molecular weight of no greater than 60 kDa from a lumen of each of the hollow fibers to the solid absorbent material(s), wherein the receptacle is configured to permit flow of a liquid along the lumen of each of the hollow fibers between the first face and the second face.

As described above and herein, the receptacles, in various configurations, comprise a plurality of hollow fibers. Each of the hollow fibers is characterized by a lumen that is shaped and sized to allow flow of a cell culture medium therethrough. The medium comprises one or more materials of biological origin, including but not limited to, cells, tissues, nutrients, supplements and feeds, amino acids, peptides, proteins, vitamins, polyamines, sugars, carbohydrates, lipids, nucleic acids, hormones, fatty acids, trace materials, and waste materials. In some embodiments, the cell culture medium comprises blood cells and the medium to be filtered is blood. In some embodiments, the cell culture medium comprises mammalian cells, chicken cells, crustacean cells, or fish cells.

In some embodiments, the waste materials are any materials that interfere with desired growth and/or desired differentiation of cells cultured in the cell culture medium. For instance, the waste materials may inhibit cell growth and/or differentiation or induce cell death. In some embodiments, the waste materials are selected amongst minerals (mainly sodium salts) and small molecules (low molecular weight molecules). By way of non-limiting examples, the waste materials include, but are not limited to, ammonia, lactate, toxins and sodium salts.

In some embodiments, a culture medium of cells or tissues is filtered and recycled, wherein tissues are cultured for antibody production. Through the filtration, the waste materials are removed from the culture medium, while the produced (or secreted) antibodies are retained in the culture medium.

In some embodiments, a culture medium of cells or tissues is filtered and recycled, wherein tissues are cultured for growth factor production. Through filtration, the waste materials are removed from the culture medium, while the produced (or secreted) growth factors are retained in the culture medium.

In some embodiments, a culture medium of cells or tissues is filtered and recycled, wherein tissues are cultured for cultured meat production in at least one container, e.g., a bioreactor. Through the filtration, the waste materials that interfere with the proper growth of the cultured meat and/or that cause cell death are removed from the culture medium, while nutrients needed for the proper growth of the cultured meat are retained in the culture medium.

To permit flow of the culture medium along the lumen, each hollow fiber is configured to have an internal diameter of at least 0.1 mm, or at least 0.5 mm, or at least 0.75 mm, up to 5 mm. In some embodiments, each hollow fiber is configured to have an internal diameter that permit flow of cells and other culture components having diameters of between 5 and 20 micrometers.

The hollow fibers may be regarded as tubular membranes having membranous or porous walls which permit irreversible passage of waste materials from the lumen to a region outside of the hollow fibers occupied by solid absorbent material(s). The irreversibility of waste material passage depends inter alia on the ability of the solid absorbent material(s) to irreversibly hold or associate to the waste materials. While absorption onto the solid absorbent material(s) may be a dynamic steady state where waste materials have a high probability of binding and a low probability of being released, based on their affinity, an insignificant flow back of waste materials may be observed. Thus, within the scope of the present disclosure, the back flow of waste materials may be as high as 50%.

The porous hollow fiber walls act to prevent nutrients and other essential materials from crossing through. This is achieved by a porosity profile selected to provide optimal pore size and pore density. Each hollow fiber may be selected to have the same porosity profile. While the pores diameters (cut-off size) may not be constant, the pores diameter should on average be selected to prevent passage of high molecular weight materials, while permitting facile and efficient passage of small molecules, i.e., low molecular weight waste materials. In some embodiments, the cut-off pore size is no greater than or smaller than 60 kDa (and different from or greater than 0 kDa). In some embodiments, the average pore diameter is such that a material having a molecular weight of between 10 and 60 kDa can pass through. In some embodiments, the average pore diameter is such that a material having a molecular weight of between 10 and 20 kDa, between 10 and 25 kDa, between 10 and 30 kDa, between 10 and 35 kDa, between 10 and 40 kDa, between 10 and 45 kDa, between 10 and 50 kDa, between 10 and 55 kDa, between 15 and 60 kDa, between 20 and 60 kDa, between 25 and 60 kDa, between 30 and 60 kDa, between 35 and 60 kDa, between 40 and 60 kDa, between 45 and 60 kDa or between 50 and 60 kDa can pass through. In some embodiments, the cutoff pore diameter is no greater than 10 kDa.

The pore density, namely the number of pores per unit surface area of the inner fiber wall, may be varied according to the porosity of the hollow fibers. In some embodiments, at least 10% of the inner fiber walls are porous. In some embodiments, up to 80% of the inner fiber walls are porous.

The cell culture medium comprises nutrients, essential materials, and waste materials, wherein separation is desired to remove the waste materials from the medium. The essential materials and nutrients are differentiated from the waste materials according to their sizes in that the waste materials are materials having molecular weights below (or no greater than) 60 kDa, whereas the essential materials and nutrients are materials having molecular weights greater than or equal to 61 kDa.

For the receptacles described above and herein, the at least one solid absorbent material is packed in each receptacle at a space between the plurality of hollow fibers. In other words, the solid absorbent material(s) may be regarded as a matrix absorbent material that occupies a volume outside and between the plurality of hollow fibers. The solid absorbent material(s) may be in a form that is capable of irreversibly binding/associating to the waste materials passing through pores of the fiber walls, as explained above and herein. The solid adsorbent material(s) may be in the form of a resin or in the form of a pellet, a granule, a capsule or in an amorphous form. Notwithstanding of the form, the solid absorbent material(s) may be any material suitable for waste management.

In some embodiments, the at least one solid absorbent material is selected to have a binding capacity of about 5-100 mg per gram of solid absorbent material. For example, for ammonia, clinoptilolite zeolite may be used with a binding capacity ranging between 9 to 20 mg NH₄+/g zeolite. Bentonite can be used for binding ammonia at rates of about 5 mg NH₄+/g zeolite. Lactate binding may be achieved with Amberlite® IRA-400, which exhibits a binding capacity ranging between 20 to 40 mg lactate/g resin.

Notwithstanding, the solid absorbent material(s) may be selected from or may comprise one or more of a microporous aluminosilicate material, an activated carbon, an ion-exchange resin, a charged polymer, a silica gel, a clay material, and a resin material. In some embodiments, the solid absorbent material is a resin material selected from the group consisting of polyester resins, phenolic resins, alkyd resins, polycarbonate resins, polyamide resins, polyurethane resins, silicone resins, epoxy resins, polyethylene resins, polypropylene resins, acrylic resin resins and polystyrene resins.

In some embodiments, the waste materials comprise ammonia and the solid absorbent material or the waste treatment material comprises an aluminosilicate material. In some embodiments, the solid absorbent material is or comprises clinoptilolite.

In some embodiments, the waste molecules comprise lactate and the solid absorbent material or the waste treatment material comprises an ion-exchange resin. In some embodiments, the solid absorbent material is Amberlite®. In some embodiments, the solid absorbent material is or comprises Amberlite® IRA-400.

In some embodiments, the waste molecules comprise amphiphilic toxins and the solid absorbent material or the waste treatment material comprises carbon. In some embodiments, the solid absorbent material is or comprises activated carbon.

In some embodiments, the waste molecules comprise excess sodium ions and the solid absorbent material or the waste treatment material comprises ion-exchange resin. In some embodiments, the solid absorbent material is Amberlite®. In some embodiments, the solid absorbent material is or comprises Amberlite® 252RFH.

In some embodiments, the solid absorbent material is a zeolite. In some embodiments, the amount of zeolite used is between 7.5 to 600 g zeolite per liter volume of a bioreactor. In some embodiments, zeolite is used for removing ammonia from the culture medium.

In some embodiments, the solid absorbent is Amberlite IRA-400. In some embodiments, the amount of Amberlite IRA-400 used is between 750 to 54,000 g Amberlite IRA-400 per liter volume of a bioreactor. In some embodiments, Amberlite IRA-400 is used for removing lactate.

The receptacles described above and herein may be used in conjunction with an alternating tangential flow (ATF) system, a Tangential Flow Filtration (TFF) system, or a fed-batch culturing system. Thus, the receptacle may be directly or indirectly connected to at least one liquid flow adaptor, configured to connect the receptacle to an alternating tangential flow (ATF) system, to a Tangential Flow Filtration (TFF) system, or to a fed-batch culturing system.

In some embodiments, the receptacle is directly or indirectly connected to a flow adaptor configured to connect the receptacle to a hemoperfusion system.

In some embodiments, the receptacle is configured to recycle up to 1000 liters of liquid.

Another aspect of the present disclosure provides a system for filtering a cell culture medium. Such system comprises at least one receptacle as described above and herein; means for flowing a medium through the plurality of hollow fibers in the receptacle; means for circulating the medium; and a bioreactor.

In some embodiments, the at least one receptacle is disposable.

In some embodiments, the system is free of an absorption column.

In some embodiments, the means for flowing a medium comprises or is a pump.

In some embodiments, the system further comprises at least one sensor configured to record values of at least one parameter related to the flow of the medium through the receptacle and/or content of the medium.

In some embodiments, the system may further comprise a controller electrically connected to the pump and the at least one sensor, wherein the controller is configured to activate said pump based on signals received from the at least one sensor.

The system described above and herein may further comprise at least one flow adaptor configured to fluidically connect one or more receptacles to a recycling system. By way of non-limiting examples, the recycling system may be an alternating tangential flow (ATF) system, a Tangential Flow Filtration (TFF) system, a fed-batch culturing system or any variation thereof. In some embodiments, two or more receptacles are fluidically connected to the recycling system. In some embodiments, each of the receptacles is configured to treat different waste materials. In some embodiments, the two or more receptacles are fluidically connected in a row or in parallel to each other.

In some embodiments, each of the two or more receptacles in the system described above and herein is configured to treat, e.g., to remove and/or deactivate, two or more different waste materials. By way of a non-limiting example, the waste materials comprise both ammonia and lactate. In some embodiments, each of the receptacles comprises a mixture of solid absorbing materials for treatment of the two or more different waste materials. In some embodiments, each solid absorbing material in the mixture is packed separately at different compartments inside the receptacle. In some embodiments, the different compartments are fluidically connected in a row or in parallel within the receptacle.

In some embodiments, the receptacle is configured to have a dead volume of less than 100 ml, e.g., less than 50 ml, less than 20 ml, less than 10 ml, less than 5 ml or any intermediate, smaller or larger volume.

In some embodiments, the receptacle is disposable. In some embodiments, a single receptacle is configured to recycle up to 1000 liters of liquid, e.g., up to 500 liters, up to 100 liters or any intermediate, smaller or larger volume of liquid. In some embodiments, the receptacle has a mean time before failures of up to 30 days, when recycling 500 liters of cell culture medium per day.

In some embodiments, the system comprises a passive flow receptacle. In some embodiments, the passive flow receptacle is configured to recycle the culture medium by osmosis or diffusion. In some embodiments, the passive flow receptacle is an add-on or removable device to a cell culture container, e.g., a cell culture plate, a cell culture flask, or a cell culture bioreactor. In some embodiments, the passive flow receptacle is submerged at least partly in a culture medium within a cell culture container such as a bioreactor. In some embodiments, the passive flow receptacle is removably attached to a wall of the cell culture container. Alternatively, the passive flow receptacle is non-removably attached to and is an integral part of the cell culture container.

In the system described above, the culture medium filtering or recycling device is in a flow communication with a cell culture container, e.g., a cell culture plate, a cell culture flask or a bioreactor. The culture medium from the container flows through the filtering or recycling device. In some embodiments, the culture medium flowing into the filtering or recycling device comprises cells or tissues cultured in suspension. In some embodiments, the culture medium flowing into the filtering or recycling device comprises waste molecules and nutrients that are needed for proper growth and/or differentiation of the cells. By way of non-limiting examples, the nutrients include, but are not limited to, at least one of proteins, hormones, and growth factors.

After the filtration, the recycled medium exiting the filtering or recycling device comprises less than 30%, e.g., less than 20%, less than 10%, less than 5%, less than 2% or any intermediate, smaller or larger percentage value of waste molecules compared to the amount of waste molecules in the culture medium entering the filtering or recycling device. In some embodiments, the recycled medium exiting the recycling device comprises more than 60%, e.g., more than 70%, more than 80%, more than 90%, more than 95% or any intermediate, smaller or larger percentage value of selected nutrients compared to the amount of the selected nutrients in the culture medium entering the filtering or recycling device.

In some embodiments, the cell culture medium is a suspension containing animal cells that is perfused using a pump into the hollow fibers. The pump may be a positive displacement pump that works to push the suspension through the hollow fiber or to alternate between pushing the suspension into the hollow fiber and drawing it out into the bioreactor. In some embodiments, the cells are retained with the nutrients due to their sizes. In some embodiments, animal cells are retained in the bioreactor using a filter and only the culture medium is introduced to the hollow fibers.

A further aspect of the present disclosure provides a method or process for filtering a cell culture medium. Such method or process comprises flowing the cell culture medium through any of the receptacles described above and herein, and passing waste molecules through the walls of the plurality of hollow fibers to at least one solid absorbent material present at a space between the plurality of hollow fibers, while retaining the nutrients in the lumen. Through the filtration, the solid absorbent material is in a liquid environment having a pH≥7.

In some embodiments, the receptacle comprises a plurality of hollow fibers, each of which has a first opening and optionally a second opening and a lumen defined by the walls of the hollow fibers, permitting flow of a cell culture medium comprising waste materials and nutrients through the first opening. The fiber walls have a porosity profile that permits passage of the waste materials through the lumen to the solid absorbent material(s) present at a space between the plurality of hollow fibers.

In some embodiments, the method of process further comprises collecting the cell culture medium from the first or second opening, if present, and re-flowing it through the hollow fibers one or more times, thereby recycling the cell culture medium.

The systems or processes described above and herein do not require complicated feedback mechanisms. In some embodiments, the recycling of a cell culture medium is performed in an open loop process, that is, without taking into consideration the level of the waste materials in the cell culture medium. In some embodiments, the system is activated by a single pump for both removal of the waste materials from the culture medium and retaining of the desired nutrients in the culture medium. The pump may be activated while keeping desired levels of nutrients and proteins in the culture medium. Alternatively, the recycling does not need active pumping of the culture medium into a receptacle and is based on passive penetration of the waste materials through the fiber walls towards the solid absorbent material. In some embodiments, a culture medium recycling is performed by actively pumping the cell culture medium through the fibers. In some embodiments, an active flow of the cell culture medium causes waste materials to penetrate through the fiber walls, while keeping nutrients in the cell culture medium. In some embodiments, a pressure of the culture medium flowing into the receptacle is up to 6 Bar, or up to 5 Bar, up to 4 Bar, or any intermediate, smaller or larger pressure value.

### Example 1: General Recycling Process

Cells and/or tissues cultured in a container secrete waste molecules such as ammonia, lactate, and amphiphilic toxins. Additionally, the waste molecules accumulate in the cell culture media during the growth of the cells and/or tissues. The waste molecules, or accumulation thereof, have negative effects on the culturing of the cells and/or tissues. For instance, the waste molecules inhibit growth and/or differentiation of the cultured material. In general, the cell culture medium is recycled after the waste molecules are treated, e.g., removed or deactivated, while keeping proteins and other molecules that are important for growth and differentiation within the culture medium.

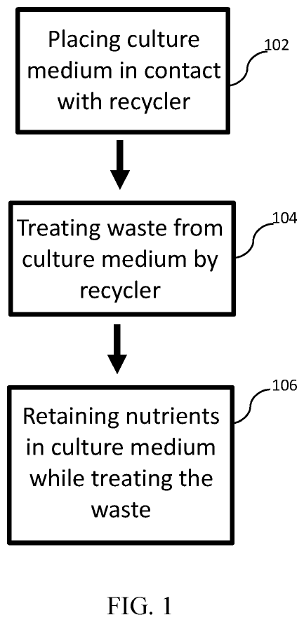

Referring to FIG. 1, a general process for recycling cell culture media is depicted. In this process, a cell culture medium is placed in contact with a culture medium recycling device, e.g., a recycler, at block 102. The culture medium is actively delivered by a pump into the recycler, or alternatively, the culture medium passively enters into the recycler by diffusion or osmosis. The recycler is placed in the culture medium, e.g., inside a container used to grow and/or to differentiate cells or tissues, or alternatively, the recycler is an integral part of the container. The container may comprise at least one cell culture plate, at least one flask configured to culture cells and/or tissues, and/or at least one bioreactor.

Generally, waste molecules from the cell culture medium are treated by the recycler, at block 104 (FIG. 1). The recycler deactivates the waste molecules, e.g., reduces or eliminates an effect of the waste molecules on the growth and/or differentiation of the cultured cells and/or tissues. Following deactivation, the waste molecules remain in the culture medium or are transferred back to the culture medium. Alternatively, the recycler removes the waste molecules from the culture medium, e.g., by adsorbing or absorbing the waste molecules from the culture medium.

While the waste molecules are treated, selected molecules required for the growth and/or differentiation of the cultured cells and/or tissues, e.g., nutrients, are retained in the culture medium, at block 106 (FIG. 1). The recycler retains the nutrients in the culture medium by preventing the nutrients from contacting the materials used for treating the waste molecules. To do so, the recycler may selectively prevent the nutrients from contacting the materials by a filtering membrane configured to allow passage of selected molecules from the culture medium to the materials used to treat the waste molecules. Alternatively, the recycler may retain the nutrients in the culture medium by selecting materials for the treatment of the waste molecules that are inert, e.g., do not bind and/or modify at least some specific nutrients.

### Example 2: Cell Culture Medium Recycling

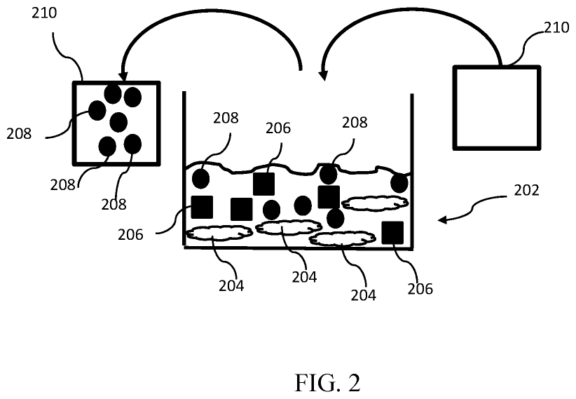

Referring to FIG. 2, a cell culture medium recycling process is depicted. In this process, cells and/or tissues, e.g., cells 204, are cultured in container 202. The container 202 may comprise a cell culture plate, a cell culture flask, or a bioreactor. The cells 204 are differentiated inside the container 202 into more specialized cells and/or to form tissues which optionally include a mixture of specialized cells. Alternatively, the tissues in the container 202 may comprise cultured meat.

The cells 204 are cultured and/or differentiate in a cell culture medium inside the container 202. The cell culture medium may comprise liquid and nutrients, e.g., soluble nutrients 206. The nutrients comprise proteins such as albumin, at least one growth factor, at least one vitamin, at least one carbohydrate, at least one lipid, at least one hormone, at least one mineral, at least one trace element and/or other serum components.

The cell culture medium also comprises waste molecules 208. The waste molecules are generated during the culturing and/or differentiation of the cells and/or tissues in the container 202. The waste molecules are usually byproducts of growth and/or differentiation of the cells or tissues, which interfere with desired growth or differentiation of the cells and/or tissues. The concentration of the waste molecules increases with time in the cell culture medium as the cells and/or tissues grow and/or differentiate. The waste molecules comprise at least one protein, at least one chemical, or at least one organic molecule.

A cell culture medium recycling device, e.g., recycler 210, is placed in contact with the culture medium in the container 202, as described at block 102 of FIG. 1. The recycler 210 comprises a filter, e.g., a filtering membrane, configured to allow treatment of the waste materials 208. One of the treatments is deactivation and/or removal of selected molecules from the culture medium. In doing so, the filter of the recycler is configured to allow treatment of selected molecules based on at least one characteristic of the molecules, including, but not limited to, size, weight, and electrical affinity.

Still referring to FIG. 2, the recycler 210 selectively treats at least some of the waste molecules 208 in the cell culture medium, while retaining the nutrients 206 in the cell culture medium. The recycler 210 is also configured to prevent removal of cells 204 from the cell culture medium in the container 202 when the cells 204 are cultured in suspension.

### Example 3: Culture Medium Recycling Device

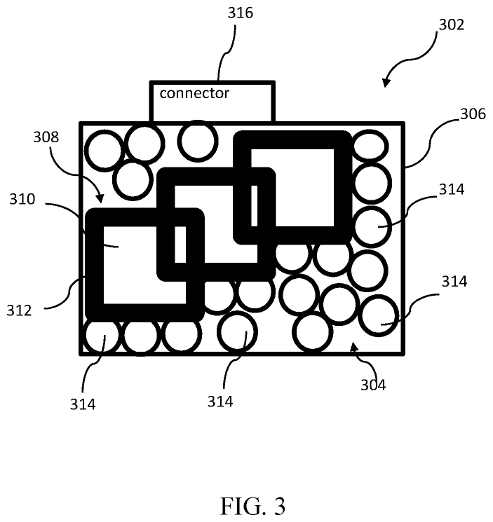

Referring to FIG. 3, a culture medium recycling device is depicted. Such device comprises a receptacle 302, which has an inner void 304 and an outer shell 306 surrounding the inner void 304. The outer shell 306 comprises one or more openings, which are shaped and sized to allow penetration of fluid such as a culture medium into and out from the receptacle 302.

The receptacle 302 comprises at least one filter, e.g., 2, 3, 4, 5, 6, 7, 10, 20, 30 or any smaller or larger number of filters within the void 304. As shown in FIG. 3, filter 308 is a hollow filter comprising an inner lumen 310, which is shaped and sized to allow flow of a culture medium through the receptacle 302. Additionally, the filter 308 comprises a membrane 312, configured to selectively allow passage of selected molecules from the inner lumen 310 of the filter 308 through the membrane 312 towards the waste treatment material 314 and/or into the receptacle void 304. The selective passage is based on at least one parameter of the molecules, including but not limited to size, weight, and affinity. By way of a non-limiting example, the membrane 312 allows passage of proteins having a molecular weight smaller than 65 kDa, 60 kDa, or any intermediate, smaller or larger value.

Another non-limiting feature of the membrane 312 is that the membrane is porous and comprises pores having a maximal size, or a maximal aperture, of about 10 kDa, or about 20 kDa, or about 30 kDa, 60 kDa, in diameter, or any intermediate, smaller or larger size. A filtering membrane with a maximal aperture of up to 60 kDa prevents passage of proteins that adsorb to solid surfaces through the pores. By way of a non-limiting example, such protein is albumin, which is a carrier protein found in cell culture media.

The membrane 312 is usually made from a material or is coated with a material that prevents attachment of cells or proteins to the membrane. The aperture of the pores is small enough to prevent proteins from passing through the pores. The prevention of protein attachment to the membrane and/or protein passage through the pores is advantageous in that it prevents clogging of the membrane pores by the cells in the culture medium.

Still referring to FIG. 3, the receptacle 302 comprises waste treatment material 314, located between the membrane 312 and the outer shell 306 of the receptacle 302. Optionally, the external surface of the membrane 312 facing the void 304 is coated with the waste treatment material 314. Alternatively, the waste treatment material 314 is located in the void 304 between adjacent filters. The waste treatment material 314 may come in contact with at least partly the external surface of the filter, e.g., the external surface of the membrane 312. The waste treatment material 314 may be packed in the form of capsules, granules or resin.

The waste treatment material 314 is configured to remove waste molecules from the culture medium and/or deactivate the waste molecules that pass through the membrane 312 and interact with the waste treatment material 314. The waste treatment material 314 adsorbs or absorbs waste molecules from the culture medium.

Further as shown in FIG. 3, the receptacle 302 comprises at least one flow path adaptor 316, optionally located in the one or more openings of the outer shell 306 thereof. The adaptor 316 is configured to connect the receptacle 302 to a culture medium flow path that interconnects a cell culture container and the receptacle. Such configuration includes, but is not limited to, connecting the receptacle, which is optionally a disposable receptacle, to an alternating tangential flow (ATF) system, a Tangential Flow Filtration (TFF) system, or a fed-batch culturing system. By way of a non-limiting example, the adaptor 316 is configured to connect the receptacle to a hemoperfusion system, wherein toxic molecules are deactivated and/or removed from the blood. By way of non-limiting example, the toxic molecules are adsorbed by the waste treatment material 314.

### Example 4: Cell Culture Medium Recycling Using Hollow Filter

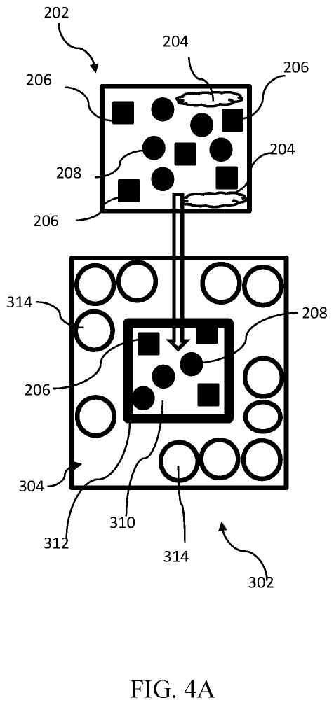

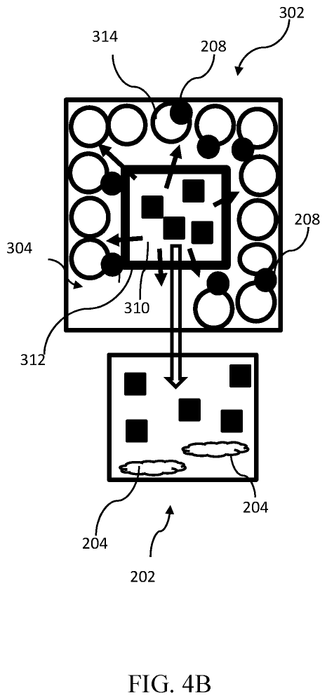

A non-limiting example of culture medium recycling process is depicted in FIGS. 4A and 4B, which uses the recycling device of FIG. 3. In this process, cells 204 are cultured in a cell culturing container 202. The cells 204 are cultured in suspension in the culture medium, or alternatively, the cells are attached to the inner walls of the container 202.

The container 202 is fluidically connected to an inner lumen 310 of at least one culture medium recycling device via at least one tube. The container 202 and the recycling device can be part of an alternating tangential flow ATF system, TFF system, or a fed-batch culturing system. Alternatively, the inner lumen 310 of the recycling device is fluidically connected to a reservoir of a hemoperfusion system.

Fluid comprising culture medium or blood flows into the inner lumen 310 of a receptacle 302 of the recycling device. The fluid flowing in the inner lumen 310 comprises nutrients 206 and waste molecules 208 (FIG. 4A). While passing through the inner lumen 310, the fluid is pushed against the membrane 312, which is configured to allow selective passage of molecules from the inner lumen 310 through the membrane 312 and then into the inner void 304 of the receptacle 302 (FIG. 4B). The selective passage is based on at least one parameter of the molecules such as size, shape, weight, and/or affinity. A non-limiting example of the affinity parameter is electrical affinity.

By way of non-limiting example, the membrane 312 comprises a plurality of pores, and the selective passage is through the pores. The membrane 312 allows selective passage, through the pores, of molecules having a molecular weight of up to 10 kDa, or up to 60 kDa, or up to 70 kDa, or any intermediate, smaller or larger weight. The selective passage allows molecules that weight less than albumin or are smaller than albumin to pass through the pores of the membrane 312.

As shown in FIG. 4B, waste molecules 208 pass through the pores of the membrane 312 into the inner void 304 of the receptacle 302. By way of non-limiting example, the inner void 304 is packed with waste treatment material 314 configured to deactivate waste molecules, adsorb waste molecules, and/or to absorb waste molecules from the culture medium entering the inner void 304.

By way of a non-limiting example, the waste treatment material 314 selectively deactivates, selectively adsorbs, and/or selectively absorbs molecules from the fluid such as waste molecules 208. The selective treatment of waste molecules can be based on selective interacting moieties, e.g., selective binding molecules covalently bound to the external surface of the waste treatment material. Such selective binding is based on the affinity properties of the waste molecules, which can be irreversible.

Also as shown in FIG. 4B, the waste treatment material 314 removes the waste molecules 208 from the fluid passing through the inner lumen 310 of the receptacle 302. Selective removal of the waste molecules 208 is based on selective passage through the membrane 312 and/or selective interaction such as binding, adsorbing, and/or absorbing with the waste treatment material 314.

Further as shown in FIG. 4B, fluid exiting the recycling device include less waste molecules compared to the fluid entering the recycling device, while the level of nutrients may be lower in the fluid exiting the recycling device relative to the nutrients content in the fluid entering the recycling device. By way of a non-limiting example, the decrease of the nutrients content may be up to 5%, up to 2%, up to 1%, up to 0.5%, up to 0.1% or any intermediate, smaller or larger percentage. It is noted that the amount of the nutrients content remains largely stable in the fluid when passing through the recycling device.

Fluid exiting the recycling device may return to the container 202 (FIG. 4B). Alternatively, when the recycling device is part of a hemoperfusion system, the fluid is transferred to a reservoir of the system and/or to a body of a subject.

### Example 5: Recycling Device Containing Hollow Fibers

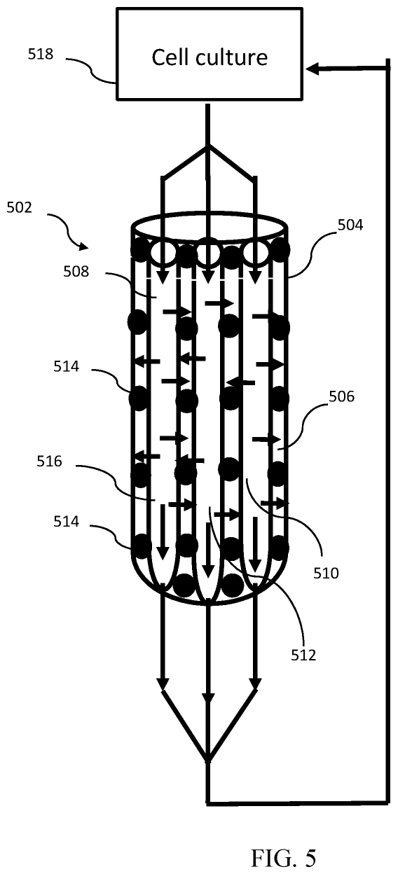

A fluid recycling device which contains one or more hollow fibers is depicted in FIG. 5. A fluid recycling device 502 comprises a receptacle 504 having an inner void 506 surrounded by a receptacle wall. The device 502 comprises hollow fibers 508, 510 and 512 in the receptacle inner void 506. The device 502 also comprises waste treatment material 514 in the inner void 506 between the hollow fibers as well as between the hollow fibers and the receptacle wall.

An external surface of the hollow fiber may be coated, at least partly, with a layer of the waste treatment material 514. Alternatively, or additionally, the waste treatment material 514 is shaped in the form of pellets, granules, capsules or resin, in the inner void 506. Optionally, the waste treatment material 514 is in direct contact with the hollow fibers 508, 510 and 512.

As shown in FIG. 5, a cell culture medium from container 518, or blood from a reservoir or a body of a subject, actively flows through an inner lumen 516 of the hollow fibers. The membrane of the hollow fibers surrounding the inner lumen 516 is configured to allow selective passage of fluid and molecules from the inner lumen 516 towards the waste treatment material 514 in the inner void 506 of the receptacle 504.

By way of a non-limiting example, a layer of the waste treatment material 514 is coated on the external surface of the hollow fiber. The hollow fiber membrane is porous and comprises a plurality of pores. The selective passage of molecules is based on the size and/or shape of the pores, and the hollow fiber membrane and/or the pores do not block or interfere with bi-directional passage of fluid through the membrane. By way of a non-limiting example, the hollow fiber membrane is configured to allow selective passage of molecules having a molecular weight of less than 10 kDa, less than 20 kDa, less than 40 kDa, less than 60 kDa, less than 70 kDa, or any intermediate, smaller or larger weight, towards the waste treatment material.

Molecules that pass through the hollow fiber membrane interact with the waste treatment material 514. The molecules are adsorbed and/or deactivated by the waste treatment material 514. When the molecules are deactivated by the waste treatment material, they return to the inner lumen 516 of the hollow fiber in a deactivated form. Alternatively, the deactivated molecules remain bound to the waste treatment material 514.

Treatment of waste molecules in the fluid is performed as fluid passes through the recycling device, e.g., through the inner lumens of the hollow fibers 508, 510 and 516. The fluid exiting the recycling device 502 is returned to the container 518 (FIG. 5). In a hemoperfusion system, blood exiting the recycling device is returned to a reservoir of the hemoperfusion system or to a body of a subject.

### Example 6: Recycling System

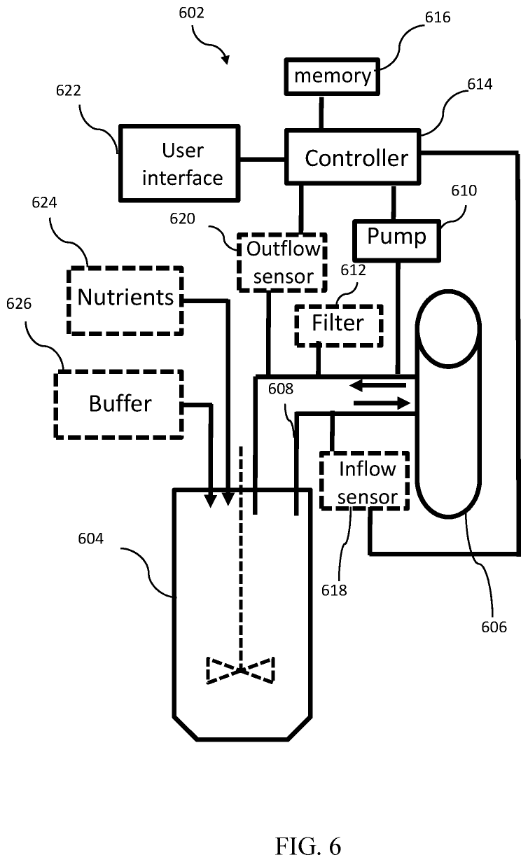

The recycling device shown in FIG. 3 and FIG. 5 may be connected to a recycling system such as an ATF system, a TFF system, or a fed-batch culturing system. The recycling system may be a closed loop system, wherein flow and/or the recycling process is controlled automatically based on signals from at least one sensor. FIG. 6 depicts such a recycling system.

A recycling system 602 comprises a fluid reservoir 604. By way of non-limiting examples, the fluid reservoir may be a body fluid reservoir such as a blood reservoir, a cell culture container comprising a cell culture plate, a cell culture flask or a bioreactor. The bioreactor may be used for culturing cells, tissues, and/or cultured meat.

The recycling system 602 also comprises a recycling device 606, which is similar to the recycling device 302 shown in FIG. 3 or the recycling device 502 shown in FIG. 5. The recycling device 606 may be configured in such a way that it can be disassembled from the system 602. Optionally, the recycling device 606 is disposable.

The recycling device 606 is fluidically connected to the reservoir 604 by at least one tube, e.g., tubing 608, configured to deliver fluid from the reservoir into the recycling device 606 and from the recycling device 606 back to the reservoir 604. By way of a non-limiting example, the recycling system 602 comprises at least one pump 610 coupled to the tubing 608. The at least one pump 610 is configured to generate active flow of liquid in the tubing 608. The at least one pump may be configured to generate pressure as measured in the inlet of the recycling device 606. By way of a non-limiting example, the recycling system comprises a single pump, allowing a simple system for recycling fluids with minimal number of elements.

The recycling system 602 may be configured to treat by removing and/or deactivating more than a single type of waste molecules. By way of non-limiting examples, the system is configured to treat at least two types of waste molecules present in the fluid. The waste molecules include, but are not limited to, ammonia and lactate. Optionally, the device 606 may be divided into different portions, each comprising a different waste treatment material targeting a different type of waste molecules. Alternatively, the device 606 may comprise a single portion with a mixture of waste treatment materials for treating a mixture of different types of waste molecules. Using a single recycling device for the treatment of several waste molecule types could increase system simplicity by using a single recycling element with a single set of activation parameters.

Alternatively, the recycling system may comprise at least one additional recycling device, e.g., an additional filter, for treating a different type of waste molecules than the device 606. The additional filter 612 comprises a different waste treatment material than the waste treatment material in the device 606, and is fluidically connected to the tubing 608. The additional filter 612 is fluidically connected to the device 606 in parallel or in a row. The at least one pump 610 actively generate fluid flow into both device 606 and the additional filter 612. Such system is useful and may increase efficiency of recycling for treatment of different types of waste molecules that have different binding affinities, and/or need to be treated by different methods.

The recycling system 602 also comprises at least one controller 614, which is functionally connected to the at least one pump 610. By way of a non-limiting example, the controller 614 is electrically connected to the pump 610. The controller 614 is configured to activate the pump 610 continuously or intermittently. The system 602 also comprises a memory 616, which is electrically connected to the controller 614. The controller 614 is configured to activate the pump 610 according to at least one activation protocol or parameters thereof, or indications, stored in the memory 616.

The recycling system may also comprise at least one sensor, e.g., an inflow sensor 618 and an outflow sensor 620, both of which are electrically connected to the controller 614. The at least one sensor is configured to record values of at least one parameter related to the fluid recycling process such as liquid flow in the tubing 608, pressure in the tubing 608, pressure in the recycling device 606, pressure in the reservoir 604, and fluid content. The at least one sensor can be at least one of a flow sensor, an optic sensor, a pressure sensor, a temperature sensor, a pH sensor and an electric sensor.

The inflow sensor 618 records values of at least one parameter related to the fluid entering the recycling device 606. The at least one parameter includes, but is not limited to, at least one of fluid temperature, fluid pH, flow speed and pressure, concentration or amount of waste molecules and/or nutrients in the fluid entering the recycling device 606. By way of non-limiting examples, the inflow sensor 618 is configured to record concentration or amount of a selected type of molecules such as ammonia molecules and lactate molecules in the liquid entering the recycling device 606.

The outflow sensor 620 records values of at least one parameter related to the fluid exiting the recycling device 606. The at least one parameter includes, but is not limited to, at least one of fluid temperature, fluid pH, flow speed and pressure, concentration or amount of waste molecules and/or nutrients in the fluid exiting the recycling device 606. By way of non-limiting examples, the outflow sensor 620 is configured to record concentration or amount of a selected type of molecules such as ammonia molecules and lactate molecules in the liquid exiting the recycling device 606.

The controller 614 is configured to determine an efficiency of the recycling process through the recycling device 606 based on signals recorded by the inflow sensor 618 and/or the outflow sensor 620. Optionally, the controller 614 calculates a score of recycling efficiency and thus determines recycling efficiency using at least one algorithm or a lookup table stored in the memory 616.

The recycling system 602 further comprises a user interface 622 electrically connected to the controller 614. The user interface 622 is configured to receive input from a user and/or to deliver an indication, e.g., a human detectable indication to a user of the recycling system 602. The controller 614 signals the user interface 622 to generate a human detectable indication, e.g., an alert signal, if the recycling efficiency is lower than a predetermined value.

By way of a non-limiting example, the controller 614 signals the user interface 622 to generate the human detectable indication if the pressure and/or flow speed of liquid exiting the recycling device 606 is at least 10%, at least 20%, at least 30%, at least 50% or any intermediate, smaller or larger percentage, lower than the pressure and/or flow speed of liquid entering the recycling device 606 or a predetermined value stored in the memory 616.

By way of a non-limiting example, the controller 614 signals the user interface 622 to generate the human detectable indication if the concentration or level of at least one type of waste molecules in liquid exiting the recycling device 606 is at least 5%, at least 10%, at least 25%, at least 30%, at least 50% or any intermediate, smaller or larger percentage value that is higher than the concentration or level of the waste molecule type in liquid entering the recycling device 606.

By way of a non-limiting example, the controller 614 signals the user interface 622 to generate the human detectable indication if the recycling device 606 needs to be replaced.

In the recycling system depicted in FIG. 6, cultured meat, cells or tissues cultured in the reservoir 604 receive nutrients and/or buffer from an external nutrients source 624 and/or an external buffer source 626. The controller 614 controls the recycling of the cell culture medium in the reservoir 604 through the recycling device 606 according to the delivery of fresh nutrients and buffer into the reservoir 604.

Fluid such as a cell culture medium, blood, and another type of body fluid, may be recycled in an open loop process using the device shown in FIG. 3 and FIG. 5, that is, without receiving feedback regarding the recycling efficiency and/or recycling process. In an open loop recycling process, the recycling device is replaced after a predetermined time period from the time the filtering membrane and/or the waste treatment material of the recycling device is first exposed to air and/or liquid. By way of non-limiting examples, the recycling device is replaced after a week, a month, 3 months, 6 months, or any intermediate, shorter or longer time period from first exposure.

In an open loop recycling process, the recycling device is optionally connected to a system that does not have a sensor or a system that includes a sensor but does not change recycling parameters through the recycling device based on signals from a sensor. Additionally, in an open loop recycling system, a user of the system does not receive an indication of changes in recycling efficiency through the recycling device.

### Example 7: Closed Loop Fluid Recycling Process

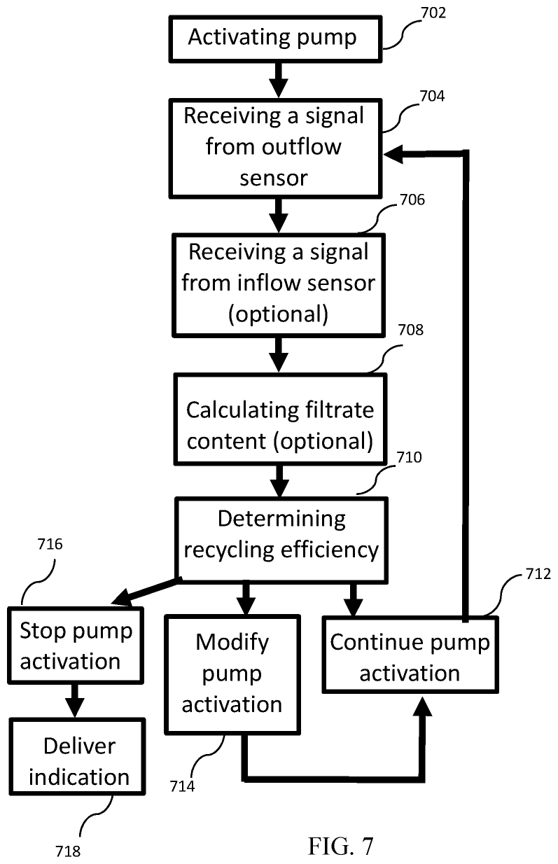

A closed loop fluid recycling process in depicted in FIG. 7. A pump is activated at block 702 by various means, e.g., by a controller (e.g., controller 614 shown in FIG. 6). The pump may be activated continuously or intermittently according to a program or at least one activation parameter or indication thereof stored in a memory (e.g., memory 616 shown in FIG. 6).

Upon activation of the pump, a signal is received from at least one outflow sensor at block 704. An outflow sensor (e.g., outflow sensor 620 shown in FIG. 6) is located at a flow path exiting a fluid recycling device (e.g., device 606 shown in FIG. 6). The outflow sensor records at least one of flow speed, fluid pressure, and fluid content, downstream the fluid recycling device. By way of non-limiting examples, the outflow sensor records levels and/or concentration of selected molecules such as waste molecules and/or nutrients molecules in the filtrate. The waste molecules include, but are not limited to, ammonia and lactate molecules.

Optionally, a signal is received from at least one inflow sensor at block 706. An inflow sensor (e.g., inflow sensor 618 shown in FIG. 6) records at least one of flow speed, fluid pressure, and fluid content, upstream the fluid recycling device. By way of non-limiting examples, the inflow sensor records levels and/or concentration of selected molecules such as waste molecules and/or nutrients molecules in fluid entering the fluid recycling device. The waste molecules include, but are not limited to, ammonia and lactate molecules.

Optionally, a content of a filtrate exiting the fluid recycling device 606 is calculated at block 708 based on the signals received from the outflow sensor. By way of non-limiting examples, the levels and/or concentration of selected molecules such as waste molecules and/or nutrients molecules in the filtrate are calculated at block 708. The waste molecules include, but are not limited to, ammonia and lactate molecules.

Fluid recycling efficiency is determined at block 710 based on the signals received from the outflow sensor at block 704 and/or the filtrate content calculated at block 708. By way of a non-limiting example, the fluid recycling efficiency is determined based on a difference between the filtrate content (i.e., the content of the fluid exiting the recycling device) and the content of the fluid entering the recycling device. Alternatively, or additionally, the fluid recycling efficiency is determined based on changes in flow speed and/or pressure between the fluid entering the recycling device and the fluid exiting the recycling device.

Upon the determination at block 710, if the fluid recycling efficiency is reduced up to 50%, e.g., up to 30%, up to 20%, up to 10%, up to 5% or any intermediate, smaller or larger percentage value, compared to the fluid recycling efficiency of an unused recycling device, then the pump activation continues at block 712 without changing pump activation parameters. If the fluid recycling efficiency is reduced more than 50%, e.g., more than 60%, more than 70%, more than 80% or any intermediate, smaller or larger percentage value, compared to the recycling efficiency of an unused recycling device, then the pump activation is modified at block 714. By way of a non-limiting example, the pump is activated to generate an increase in fluid pressure and/or an increase in flow of fluid entering the recycling device. If the pump activation is stopped at block 716, an indication such as an alert signal is delivered to a user at block 718.

### Example 8: Recycling Devices

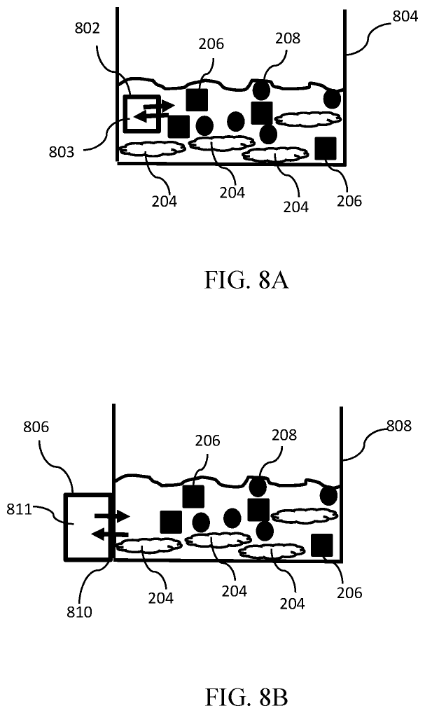

Different recycling devices may be used for the recycling process. FIG. 8A depicts a removably assembled recycling device. A cell culture recycling device is configured to treat waste molecules in fluid such as a culture medium while retaining nutrients in the culture medium. As shown in FIG. 8A, the recycling device 802, which is similar to the device 302 shown in FIG. 3, is placed inside a cell culture container 804. Alternatively, the recycling device is configured to be removably attached to a wall of the cell culture container by at least one attachment adaptor of the device. When the recycling device is attached to the container, a fluid path between the container and the device is formed, which allows the culture medium to flow in and out of the recycling device.

The removably assembled fluid recycling device is replaced after a predetermined time period. Optionally, the recycling device comprises an indicator such as a colorimetric indicator, which is configured to deliver an indication regarding the efficiency of the recycling process. By way of a non-limiting example, the recycling efficiency is indicated by a binding saturation level of the waste treatment material in the recycling device. At least one wall of the recycling device 802 comprises a filtering membrane or is coated with a filtering membrane (e.g., the filtering membrane 312 shown in FIG. 3) to allow selective penetration/passage of molecules such as waste molecules towards a waste treatment material packed in an inner lumen 803 of the recycling device.

FIG. 8B depicts an integral or non-removable recycling device. A fluid recycling device 806 is integrated with a cell culture container 808. A wall 810 between the container 808 and the device 806 comprises one or more openings that are shaped and sized to allow penetration of culture medium into the device 806 towards waste treatment material packed inside an internal lumen 811 of the device 806. The wall 810 comprises a filtering membrane (e.g., filtering membrane 312 shown in FIG. 3) or is coated at least partly with a filtering membrane (e.g., filtering membrane 312 shown in FIG. 3), which allows selective penetration of molecules such as waste molecules into the inner lumen 811.

### Example 9: Removal of Ammonia Molecules

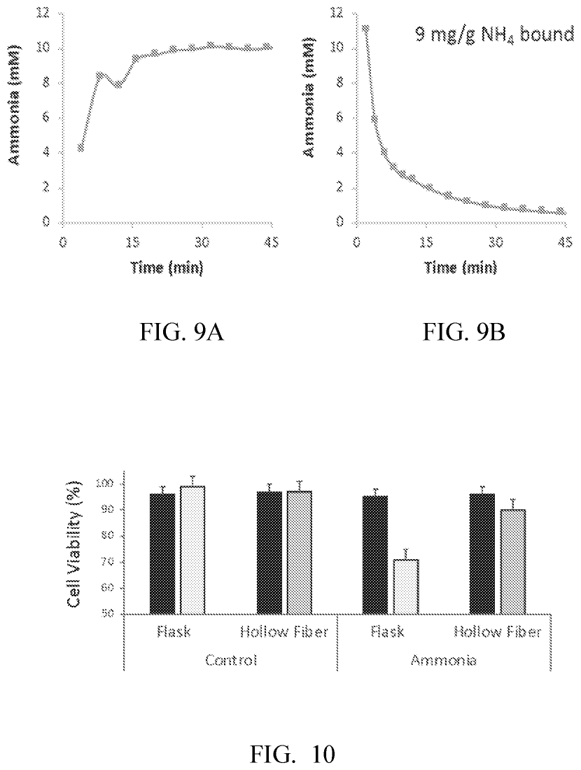

Ammonia is a byproduct of cell growth and/or differentiation, and without being bound by any theory, may be toxic to the cultured cells in high concentrations. FIG. 9A shows that ammonia molecules were accumulated in the cell culture medium over time as seen in the increase of the ammonia concentration over time.

FIG. 9B shows the active removal of ammonia from the packed hollow fiber washed by NaOH solution. In this study, ammonia was dissolved at a concentration of about 11 mM in phosphate buffer saline and passed through a hollow fiber (Xampler™ model UFP-10-C-3MA) with surface area of 140 cm² and 10 kDa pores at a flow rate of 4 ml/min. Three (3) grams of clinoptilolite (zeolite) was packed into the shell volume of the hollow fiber. It is noted that the recycling process reduced ammonia concentration from about 11 mM to about 5 mM in a matter of minutes. Clinoptilolite became saturated after 20 minutes of continuous perfusion and was then cleansed in 30 minutes, at which point 9 mg ammonia was bound to per gram of the zeolite.

### Example 10: Cell Survival

Survival of cells passing through hollow fibers was examined. As shown in Tables 1A and 1B below, spontaneously immortalized chicken cells were cultured in DMEM media with 10% fetal bovine serum and introduced into the resin-packed hollow fibers at a flow rate of 4 ml/min (Table 1A) and a flow rate of 8 ml/min (Table 1B). In both cases, the cells exhibited high viability at the point of exiting the hollow fiber and were apparently unaffected by shear rates.

TABLE 1A
Flow Rate of 4 ml/min

| | |
|---|---|
| Cell Density [million/ml] | 0.63 |
| Viability | 99% |
| Glucose [g/l] | 0.92 |
| Lactate [mmol/l] | 30 |

TABLE IB
Flow Rate of 8 ml/min

| | |
|---|---|
| Cell Density [million/ml] | 0.62 |
| Viability | 97% |
| Glucose [g/l] | 0.87 |
| Lactate [mmol/l] | 28 |

### Example 11: Ammonia Stripping

FMT-SCF2 chicken cell line was suspended at a density of 0.3 million cells/ml in baseline culture medium or culture medium spiked with 8 mM ammonia. Cell suspension containing ammonia was passed through a hollow fiber with a pore cutoff of 10 kDa whose shell was loaded with 9 g of clinoptilolite (Zeolite). As shown in Table 2 below, untreated cell suspension with 8 mM ammonia showed 71% viability within 24 hrs. In contrast, the zeolite-packed hollow fiber removed ammonia from the suspension, which reduced the concentration of ammonia from about 8.1 mM to about 5.2 mM in the suspension and thus, allowed the cells to survive with 90% viability in 24 hours.

TABLE 2

| | Baseline | Control | Hollow Fiber |
|---|---|---|---|
| Cell Density [million/ml] | 0.39 | 0.53 | 0.44 |
| Process Viability [9%] | 97% | 95% | 96% |
| Glucose [g/l] | 3.9 | 3.6 | 3.8 |
| Ammonia [mM] | 0.6 | 8.1 | 5.2 |
| Long Term Viability [%] | 97% | 71% | 90% |

FIG. 10 depicts removal of toxic concentration of ammonia from the culture medium.

Black bars represent initial cell viability for all conditions before the treatment. Yellow and green bars represent cell viability measured at the point of 24 hours after the treatment. Cells cultured in the absence of ammonia (“Control”) maintained a viability of greater than 90% in the culture shaker flasks (yellow bar) or after passing through the resin-packed hollow fiber cartridge (green bar). Cells cultured in the presence of 8 mM ammonia (“Ammonia”) showed a different behavior in that viability of the untreated cells dropped to about 71% in 24 hours (yellow bar), whereas the cells that passed through the resin-packed hollow fiber cartridge maintained a viability of greater than 90%.

### Example 12: Lactate Removal

Resins adsorb lactate through an ion exchange mechanism. In this study, lactate adsorbance capacity of different resins, e.g., Amberlite® anionic resins IRA-67, IRA-96, IR-120, and IRA-400, was examined. Sodium lactate at a concentration of about 50 mM was dissolved in DMEM culture medium with high glucose. The medium containing the lactate was mixed with 2 grams of resin in 50 ml conical tubes for 60 minutes. Analytical values (Table 3) were measured using the Flex2 Bioanalyzer (NOVA Biomedical).

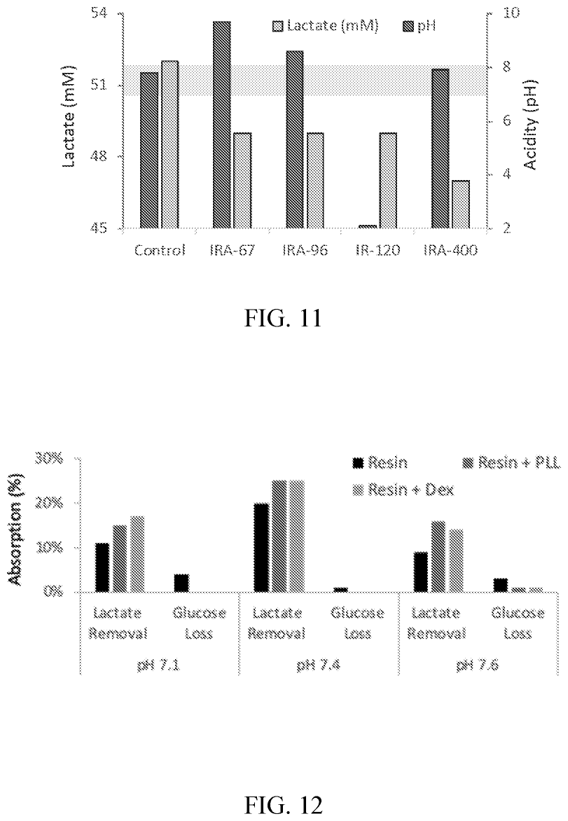

As shown in Table 3 below and in FIG. 11, Amberlite® anionic resins IRA-67, IRA-96, IR-120, and IRA-400 bound 10% of lactate in 60 minutes (2 g per 50 ml) without affecting glucose levels, pH level (except IR-120) or the mineral salt (except IR-120) composition of the culture medium.

TABLE 3

| | Glucose | Lactate | | P | Ca | Na | Mg | Cl | K |
|---|---|---|---|---|---|---|---|---|---|
| | [g/L] | [mM] | pH | [mM] | [mM] | [mM] | [mM] | [mM] | [mM] |
| Control | 3.8 | 52 | 7.8 | 1 | 1.88 | >204 | 0.8 | 127 | 5.8 |
| IRA-67 | 3.8 | 49 | 9.7 | 0.7 | 1.35 | >204 | 0.8 | 112 | 5.7 |
| IRA-96 | 3.8 | 49 | 8.6 | 1 | 1.72 | >198 | 0.8 | 116 | 5.6 |
| IR-120 | 3.7 | 49 | 2.1 | 1 | 0.06 | 110 | <0.1 | 120 | 2.2 |
| IRA-400 | 3.9 | 47 | 7.9 | 0.9 | 1.84 | >203 | 0.9 | 135 | 5.7 |

Amberlite® IRA-400 resin was coated with polycations dextran (Dex) and polylysine (PLL) to improve binding efficiency to lactate. Lactate was added at 100 mM to a cell culture medium containing 4.5 g/l of glucose. The cell culture medium was incubated with coated resin for 24 hours. Optimal lactate biding was measured at pH of 7.4, reaching about 25% of the original lactate concentration in both dextran and polylysine coatings without residual binding of glucose (FIG. 12).

It is expected that during the life of a patent maturing from this application many relevant hollow fibers and waste treatment materials will be developed; the scope of the terms hollow fibers and waste treatment material is intended to include all such new technologies a priori.

Although the present invention and its advantages have been described in detail, it should be understood that various changes, substitutions and alterations can be made herein without departing from the spirit and scope of the invention as defined in the appended claims.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A receptacle for filtering a liquid, said receptacle comprising:
(1) a plurality of hollow fibers extending the length of said receptacle; and
(2) at least one solid absorbent material occupying a space between said plurality of hollow fibers,
wherein each hollow fiber comprises at least one opening and a lumen formed by the walls thereof, said walls having a porosity profile selective to passage of waste materials contained in the liquid from the lumen to the at least one solid absorbent material, thereby filtering the liquid when it flows along the lumen.

2.-6. (canceled)

7. The receptacle of claim 1, wherein said liquid is a cell culture medium comprising blood cells, mammalian cells, chicken cells, crustacean cells, or fish cells.

8. (canceled)

9. The receptacle of claim 1, wherein said waste materials are selected from the group consisting of ammonia, lactate, toxins and sodium salts.

10. The receptacle of claim 1, wherein said liquid is a cell culture medium and the cell culture medium contains tissues cultured for antibody production, growth factor production, or cultured meat production and wherein said waste materials are removed from said cell culture medium, while produced antibodies, produced growth factors, and produced cultured meat are retained in said cell culture medium.

11.-12. (canceled)

13. The receptacle of claim 1, wherein said porosity profile is configured to provide an average pore size and pore density that permits passage of said waste materials.

14.-18. (canceled)

19. The receptacle of claim 1, wherein said at least one solid absorbent material is a microporous aluminosilicate material, an activated carbon, an ion-exchange resin, a charged polymer, a silica gel, a clay material, a resin material, or a combination thereof.

20.-22. (canceled)

23. A system for filtering a cell culture medium, said system comprising:
(1) at least one receptacle according to claim 1;
(2) means for flowing the cell culture medium through the plurality of hollow fibers in said at least one receptacle;
(3) means for circulating said cell culture medium; and
(4) a bioreactor.

24. (canceled)

25. The system of claim 23, wherein the means for flowing the cell culture medium is a pump and the system further comprises at least one sensor configured to record values of at least one parameter related to the flow of said cell culture medium through said receptacle and/or the content of said cell culture medium and a controller electrically connected to the pump and the at least one sensor, wherein said controller is configured to activate the pump based on signals received from the at least one sensor.

26. (canceled)

27. The system of claim 23, further comprising at least one flow adaptor configured to fluidically connect said at least one receptacle to a recycling system, wherein said recycling system is an alternating tangential flow (ATF) system, a Tangential Flow Filtration (TFF) system, a fed-batch culturing system, or a variation thereof.

28. (canceled)

29. The system of claim 27, wherein two or more receptacles are fluidically connected to a recycling system, wherein the two or more receptacles are configured to treat different waste materials.

30.-35. (canceled)

36. The system of claim 23, further comprising a passive flow receptacle.

37. The system of claim 36, wherein said passive flow receptacle is configured to recycle the cell culture medium by osmosis or diffusion.

38. The system of claim 36, wherein said passive flow receptacle is non-removably integrated with a cell culture container, or is removably placed inside a cell culture container or removably attached to an inner wall of a cell culture container.

39. The system of claim 38, wherein said cell culture container is a cell culture plate, a cell culture flask, or a cell culture bioreactor.

40. (canceled)

41. The system of claim 23, wherein said cell culture medium is a suspension containing animal cells, said suspension being perfused into the plurality of hollow fibers of said at least one receptacle by a pump, wherein said pump is a positive displacement pump that pushes the suspension through the plurality of hollow fibers or alternates between pushing the suspension into the plurality of hollow fibers and drawing the suspension out into the bioreactor.

42. (canceled)

43. A method for filtering a cell culture medium, said method comprising:
(1) flowing the cell culture medium through a receptacle, wherein said receptacle comprises a plurality of hollow fibers each having at least one opening and a lumen defined by the walls thereof, wherein said cell culture medium comprises waste molecules and nutrients; and
(2) passing waste molecules through the walls of the plurality of hollow fibers to at least one solid absorbent material present outside the lumen and at a space between the plurality of hollow fibers, while retaining the nutrients in the lumen, thereby filtering the cell culture medium, wherein said at least one solid absorbent material is in a liquid environment having a pH≥7.

44. The method of claim 43, further comprising collecting said cell culture medium from said at least one opening and re-flowing it through the plurality of hollow fibers one or more times, thereby recycling the cell culture medium.

45. (canceled)

46. (canceled)

47. (canceled)

48. (canceled)

49. (canceled)

50. The method of claim 43, wherein the cell culture medium is used to grow cultured meat.

51. A method for producing cultured tissues, said method comprising:
(1) culturing tissues in a cell culture medium comprising nutrients and waste molecules; and
(2) filtering the cell culture medium according to the method of claim 43 to reduce the amount of waste molecules from the cell culture medium.

52. The method according to claim 51, wherein the cultured tissues are used to produce cultured meat.
